(19) United States
(12) Patent Application Publication
(10) Pub. No.: US 2012/0071673 A1
(43) Pub. Date: Mar. 22, 2012

(54) SYNTHESIS OF (S)-N-[2-(1,6,7,8-TETRAHYDRO-2H-INDENO-[5,4-B]FURAN-8-YL)ETHYL]PROPIONAMIDE

(75) Inventor: Jerome Cluzeau, Ljubljana (SI)

(73) Assignee: LEK Pharmaceuticals D.D., Ljubljana (SI)

(21) Appl. No.: 13/148,923

(22) PCT Filed: Feb. 11, 2010
(86) PCT No.: PCT/EP2010/051693

§ 371 Date: Sep. 30, 2011

(30) Foreign Application Priority Data
Feb. 12, 2009 (EP) 09152662.4

(51) Int. Cl. C07D 307/93

(52) U.S. Cl. 549/458

(57) ABSTRACT

The present invention relates in general to the field of organic chemistry and in particular to the preparation of (S)—N-[2-(1,6,7,8-tetrahydro-2H-indeno-[5,4-b]furan-8-yl)ethyl]propionamide, i.e. ramelteon, and analogues thereof.

### FIELD OF THE INVENTION

The present invention relates in general to the field of organic chemistry and in particular to the preparation of (S)—N-[2-(1,6,7,8-tetrahydro-2H-indeno-[5,4-b]furan-8-yl)ethyl]propionamide, i.e. ramelteon.

### BACKGROUND OF THE INVENTION

Ramelteon, (S)—N-[2-(1,6,7,8-tetrahydro-2H-indeno-[5,4-b]furan-8-yl)ethyl]propionamide, is a melatonin receptor agonist with both high affinity for melatonin MT1 and MT2 receptors and selectivity over the MT3 receptor. Ramelteon demonstrates full agonist activity in vitro in cells expressing human MT1 or MT2 receptors, and high selectivity for human MT1 and MT2 receptors compared to the MT3 receptor. The activity of ramelteon at the MT1 and MT2 receptors is believed to contribute to its sleep-promoting properties, as these receptors, acted upon by endogenous melatonin, are thought to be involved in the maintenance of the circadian rhythm underlying the normal sleep-wake cycle.

The synthesis of ramelteon is disclosed in EP885210B1, EP1792899A1 and J. Med Chem. 2002, 45, 4222-4239. Ramelteon is synthesized in two parts; first the synthesis of the tricyclic core with the key intermediate 6,7-dihydro-1H-indeno[5,4-b]furan-8(2H)-one is performed in six or seven steps and th the side chain with the introduction of the chirality and amide function is performed in four steps. The synthesis uses 2,3-benzofuran as starting material and in several steps involves the use of small to large excess of halogenated reagents and in last asymmetric hydrogenation step high pressure of hydrogen (around 5 MPa) is used.

International patent application WO2008/106179 A1 discloses a ten step synthesis of ramelteon via alternative intermediates. The synthesis uses excess of halogenated reagents, as well as reagents such as liquid ammonia and borontribromide. In asymmetric hydrogenation step pressurized hydrogen is used.

International patent application WO2008/151170 A2 describes the preparation of ramelteon via key intermediate 6,7-dihydro-1H-indeno[5,4-b]furan-8(2H)-one which is in six steps transformed to ramelteon. Excess of halogenated reagents and reagents such as liquid ammonia and borontrifluoride are used. The synthesis involves additional step of resolving a desired enantiomer by salt formation.

There is a need in the art for new efficient processes for the preparation of ramelteon.

### SUMMARY OF THE INVENTION

The present invention provides the following items including main aspects and preferred embodiments, which respectively alone and in combination particularly contribute to solving the above object and eventually provide additional advantages:
1. A process for preparing the compound of formula VIII

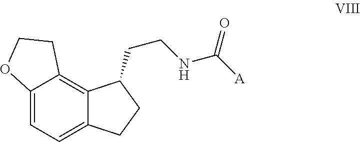

wherein A is selected from the group consisting of linear C₁-C₅-alkyl, branched C₁-C₅-alkyl, ethenyl and ethynyl, comprising the steps of:
a.) providing a compound of formula VIIa

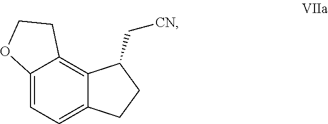

and
b.) converting the cyano group of the compound of formula VIIa into C₁-C₅-alkanamide, propenamide or propynamide group bonded to the carbon atom of the cyano group to give the compound of formula VIII.

Suitably, the conversion step b.) is carried out by reaction of the compound of formula VIIa with a compound selected from the group consisting of C₁-C₅-alkananhydrides; acryl anhydride; propargyl anhydride; and mixtures of acetanhydride and C₁-C₅-alkanoic acids, acrylic or propargylic acid.
2. The process according to item 1, wherein A is selected from the group consisting of linear C₁-C₅-alkyl and branched C₁-C₅-alkyl, and wherein in step b.) the reaction of the compound of formula VIIa is correspondingly carried out with a linear or branched C₁-C₅-alkananhydride.
3. The process according to item 1 or 2, wherein conversions step b.) involves reduction.
4. The process according to item 3, wherein the reduction involves using hydrogen in the presence of catalyst, preferably wherein the catalyst is Raney-Ni.
5. The process according to any one of the preceding items, wherein in step b.) reduction is carried out under hydrogen pressure conditions of ≦10 MPa, preferably ≧0.5 MPa.
6. A process according to any one of the previous items for preparing the ramelteon with formula VIIIa

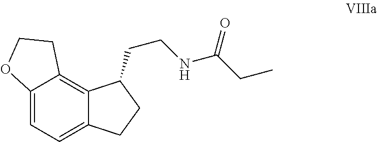

comprising the steps of:
   a.) providing a compound of formula VIIa

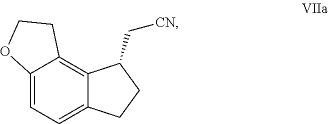

and
b.) converting the cyano group of the compound of formula VIIa into propanamide group bonded to the carbon atom of the cyano group to give the compound of formula VIIIa.
7. A process according to item 6, wherein the step of converting the cyano group of the compound of formula VIIa into propanamide group bonded to the carbon atom of the cyano group to give the compound of formula VIIIa is carried out with hydrogen and propionic anhydride in the presence of catalyst.
8. The process according to any one of the preceding items, wherein step b.) involves both a reaction of the compound of formula VIIa with said corresponding reactant and a reduction to be performed in one pot to give the respective compound of formula VIII or VIIIa.
9. A process for preparing a compound of formula VII comprising the steps of:
   a.) providing a compound of formula VI:

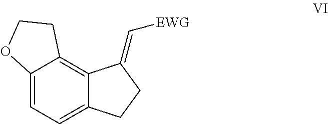

wherein EWG means an electron withdrawing group; and
b.) performing asymmetric reduction reaction of the compound of formula VI in the presence of metal-(optically active posphine)-complex catalyst, wherein metal is preferably selected from the group consisting of Cu, Co, Ni, Rh, Ru, Pd and Ir, more preferably metal is Cu, wherein said catalyst particularly is a copper-(optically active phosphine)-complex catalyst, to give the compound of formula VII:

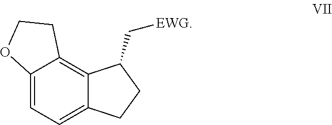

10. The process according to item 9, wherein asymmetric reduction is performed in the presence of hydride source, preferably using polymethylhydrosiloxane.
11. The process according to any one of items 9-10, wherein said metal-(optically active phosphine) complex catalyst is prepared from the corresponding metal, preferably copper, and ferrocenyl phosphines selected from the compounds having formula:

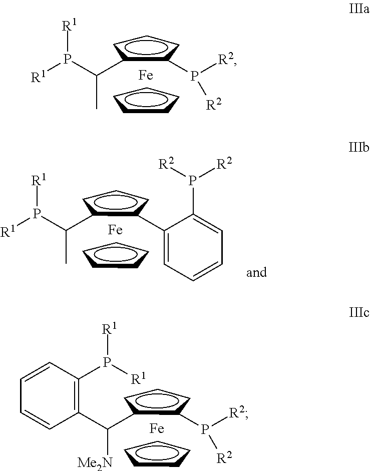

wherein R¹ and R² are independently selected from the group consisting of independently substituted or unsubstituted alkyl, cycloalkyl, aryl heteroaryl, arylalkyl and heteroarylalkyl group.

12. The process according to any one of items 9-11, further comprising subjecting the compound of formula VII to further synthetic steps to yield a compound having the formula VIII set forth below:

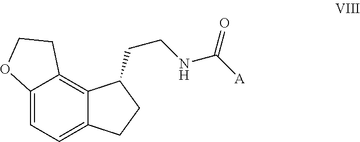

wherein A is selected from the group consisting of linear C₁-C₅-alkyl, branched C₁-C₅-alkyl, ethenyl and ethynyl.
13. The process according to item 12, wherein the further synthetic steps yield ramelteon (VIIIa).
14. The process according to any one of items 9-12, wherein the EWG group is selected from nitrile (CN), halogens (F, Cl, Br and I, preferably F and Cl), carboxylic acid (CO₂H), carboxylic acid esters (CO₂R⁷, wherein R⁷ is selected from the group consisting of substituted or unsubstituted alkyl, cycloalkyl and arylalkyl group) and amides (CONR⁸R⁹, wherein R⁸ and R⁹ are the same or different and respectively denote H, substituted or unsubstituted alkyl, cycloalkyl, [wherein “alkyl” may preferably denote C₁ to C₆ alkyl, more preferably R⁸ and R⁹ are both H].
15. The process according to any one of items 9-14, wherein the EWG group is CN.
16. The process according to items 1-8, wherein said compound of formula VIIa is prepared by a process comprising subjecting a compound of formula VIa:

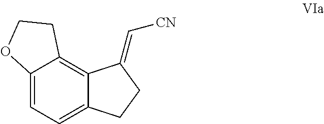

to asymmetric reduction in the presence of catalyst and a hydrogen source, wherein said catalyst is an optically active metal complex comprising a metal selected from the group of transition metals, preferably selected from the group consisting of Cu, Co, Ni, Rh, Ru, Pd and Ir, and chiral ligand.
17. The process according to item 16, wherein said catalyst is a metal-(optically active posphine)-complex, wherein metal is preferably Cu, Co, Ni, Rh, Ru, Pd or Ir, more preferably Cu, wherein said catalyst particularly is a copper-(optically active phosphine)-complex catalyst, and the hydrogen source is a hydride source, preferably hydride source is polymethylhydrosiloxane.
18. A process according to item 17, wherein said metal-(optically active phosphine)-complex catalyst is prepared from the corresponding metal, preferably copper, and ferrocenyl phosphines selected from the compounds having formula:

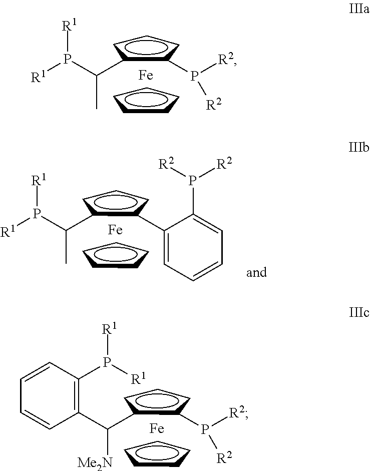

wherein R¹ and R² are independently selected from the group consisting of independently substituted or unsubstituted alkyl, cycloalkyl, aryl heteroaryl, arylalkyl and heteroarylalkyl group [wherein “alkyl” may preferably denote C₁ to C₆ alkyl].
19. The process according to any one of items 9-18, wherein the compound of formula VI is prepared by a process comprising reacting compound of formula V

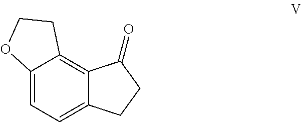

with a compound of formula (R³O)₂POCH₂(EWG), wherein EWG has the same meaning as defined in item 10 and preferably is CN, and R³ is selected from the group consisting of substituted or unsubstituted alkyl, cycloalkyl and arylalkyl group [wherein “alkyl” preferably denotes C₁ to C₆ alkyl].
20. The process according to previous item wherein the compound of formula V is prepared by a process comprising the steps of:
   a.) preparing a compound of formula II by reacting compound of formula I with vinyl acetate;
   b.) preparing a compound of formula Ill by reacting a compound of formula II with primary amine;
   c.) reacting a compound of formula Ill with paraformaldehyde in the presence of ammonium salt, R⁴R⁵NH₂⁺X⁻, (wherein R⁴ and R⁵ are each independently selected from alkyl, cycloalkyl, aryl, arylalkyl and arylcycloalkyl; and X is halogen, BF₄, PF₆, H₂PO₄ or R⁶CO₂, wherein R⁶ is one of alkyl, aryl, polyhaloalkyl) in organic solvent [wherein “alkyl” may preferably denote C₁ to C₆ alkyl];
   d.) contacting the solution from step c) with strong inorganic acid

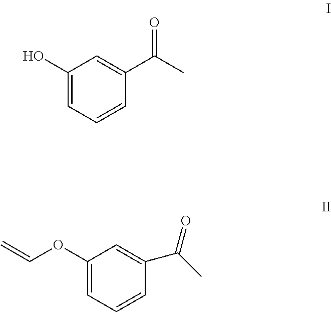

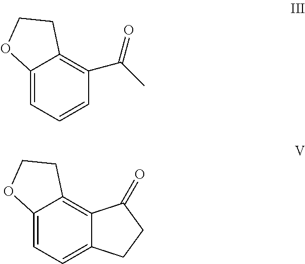

21. A compound of formula VIIa

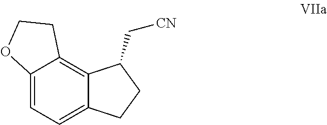

22. Use of compound (S)-2-(2,6,7,8-tetrahydro-1H-indeno[5,4-b]furan-8-yl)acetonitrile (VIIa) for the preparation of
   a compound of formula VIII

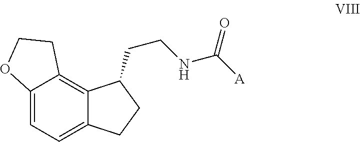

wherein A is selected from the group consisting of linear C₁-C₅-alkyl, branched C₁-C₅-alkyl, ethenyl and ethynyl.
23. Use of compound (S)-2-(2,6,7,8-tetrahydro-1H-indeno[5,4-b]furan-8-yl)acetonitrile (VIIa) for the preparation of ramelteon (VIIIa).
24. A process for preparing ramelteon (VIIIa) comprising the steps of:
   a.) preparing the compound of formula VIIa by a process comprising subjecting a compound of formula VIa to asymmetric reduction in the presence of the copper-(optically active phosphine)-complex catalyst and hydride source, wherein said copper-(optically active phosphine)-complex catalyst is prepared from copper and ferrocenyl phosphines selected from the compounds having formula:

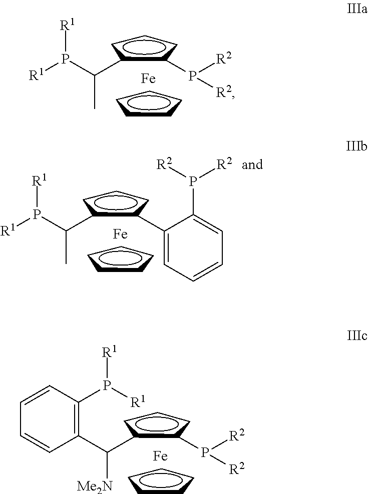

wherein R¹ and R² are independently selected from the group consisting of independently substituted or unsubstituted alkyl, cycloalkyl, aryl heteroaryl, arylalkyl and heteroarylalkyl group [wherein “alkyl” may preferably denote C₁ to C₆ alkyl]; and
b.) reacting a compound of formula VIIa with hydrogen and propionic anhydride in the presence of catalyst, wherein said catalyst is Raney-Ni, to yield a compound of formula VIIIa

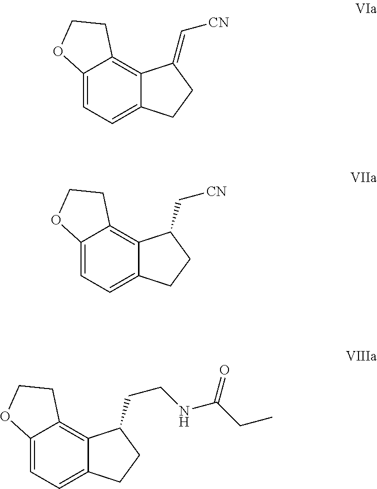

25. A process for preparing ramelteon (VIIIa) comprising the steps of:
   a.) reacting compound of formula V with cyanomethanephosphonate of formula (R³O)₂POCH₂CN, wherein R³ is selected from the group consisting of substituted or unsubstituted alkyl, cycloalkyl and arylalkyl group; to yield a compound of formula VIa
   b.) preparing the compound of formula VIIa by a process comprising subjecting a compound of formula VIa: to asymmetric reduction in the presence of the copper-(optically active phosphine)-complex catalyst and hydride source, wherein said copper-(optically active phosphine)-complex catalyst is prepared from copper and ferrocenyl phosphines selected from the compounds having formula:

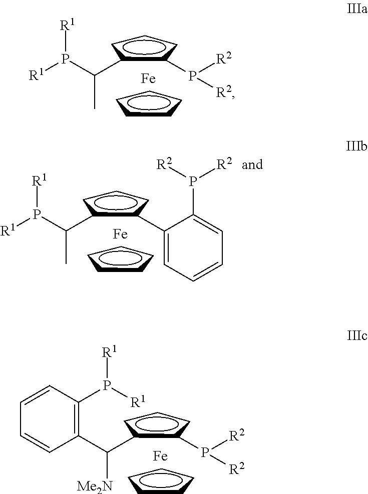

wherein R¹ and R² are independently selected from the group consisting of independently substituted or unsubstituted alkyl, cycloalkyl, aryl heteroaryl, arylalkyl and heteroarylalkyl group [wherein “alkyl” may preferably denote C₁ to C₆ alkyl]; and
c.) reacting a compound of formula VIIa with hydrogen and propionic anhydride in the presence of catalyst, wherein said catalyst is Raney-Ni, to yield a compound of formula VIIIa

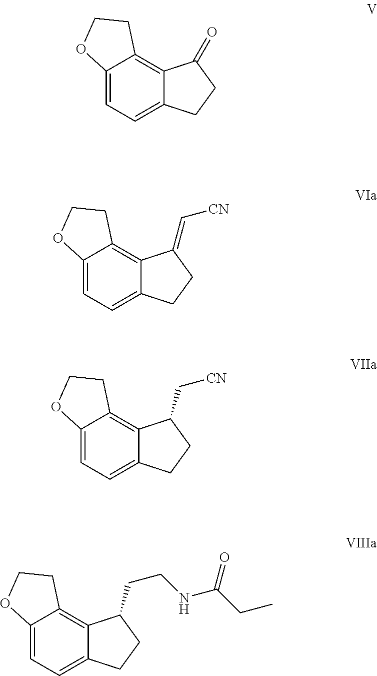

26. A process for preparing ramelteon (VIIIa) comprising the steps of:
   a.) preparing a compound of formula II by reacting compound of formula I with vinyl acetate;
   b.) preparing a compound of formula III by reacting a compound of formula II with primary amine;
   c.) reacting a compound of formula III with paraformaldehyde in the presence of ammonium salt, R⁴R⁵NH₂⁺X⁻, (wherein R⁴ and R⁵ are each independently selected from alkyl, cycloalkyl, aryl, arylalkyl and arylcycloalkyl; and X is halogen, BF₄, PF₆, H₂PO₄ or R⁶CO₂, wherein R⁶ is one of alkyl, aryl, polyhaloalkyl) in organic solvent [wherein “alkyl” may preferably denote C₁ to C₆ alkyl];
   d.) contacting the solution from step c.) with strong inorganic acid and obtaining compound of formula V;
   e.) reacting compound of formula V with cyanomethanephosphonate of formula (R³O)₂POCH₂CN, wherein R³ is selected from the group consisting of substituted or unsubstituted alkyl, cycloalkyl and arylalkyl group [wherein “alkyl” may preferably denote C₁ to C₆ alkyl]; to yield a compound of formula VIa;
   f.) preparing the compound of formula VIIa by a process comprising subjecting a compound of formula VIa: to asymmetric reduction in the presence of the copper-(optically active phosphine)-complex catalyst and hydride source, wherein said copper-(optically active phosphine)-complex catalyst is prepared from copper and ferrocenyl phosphines selected from the compounds having formula:

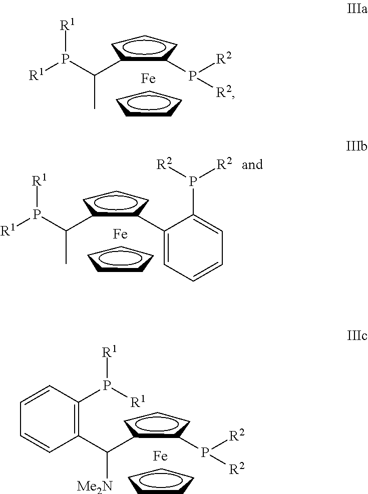

wherein R¹ and R² are independently selected from the group consisting of independently substituted or unsubstituted alkyl, cycloalkyl, aryl heteroaryl, arylalkyl and heteroarylalkyl group [wherein “alkyl” may preferably denote C₁ to C₆ alkyl]; and
g.) reacting a compound of formula VIIa with hydrogen and propionic anhydride in the presence of catalyst, wherein said catalyst is Raney-Ni, to yield a compound of formula VIIIa

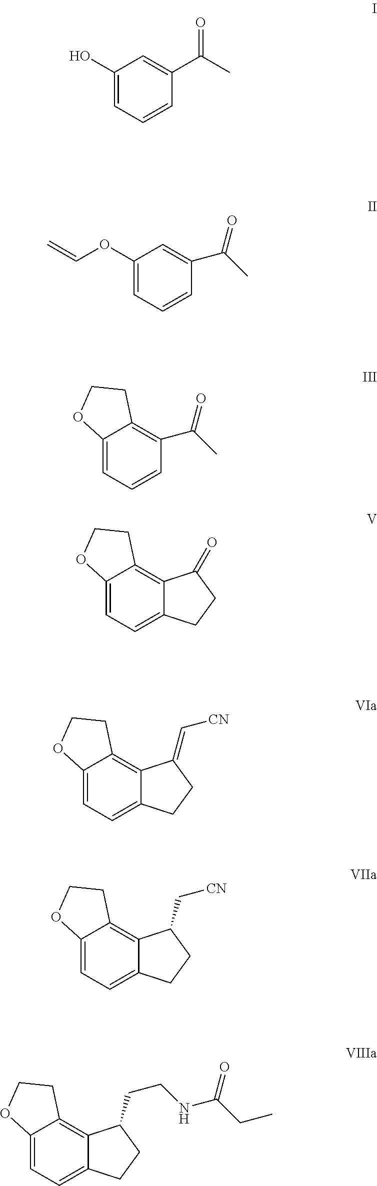

27. A process for the preparation of a pharmaceutical composition comprising ramelteon (VIIIa) as active ingredient, comprising the steps of:
   preparing ramelteon (VIIIa) according to the process according to any one of the items 6-8, 13 and 24-26, and
   admixing the thus prepared ramelteon (VIIIa) with at least one pharmaceutically acceptable excipient.

The invention solves the problem of long and tedious synthesis of ramelteon. By applying novel key steps it is possible to obtain ramelteon in a short and efficient route with yields that are industrially applicable and competitive. Compared to prior art processes reduced amounts of halogenated reagents can be used (if needed at all), and toxic and/or hazardous reagents such as liquid ammonia, borontrifluoride and borontribromide can be avoided. By adopting a relevant asymmetric reduction step applied to an adequately chosen compound, which can be performed advantageously under atmospheric pressure or low pressure as desired, the use of H₂ under hazardous high pressure conditions can be avoided. In addition a step of resolving a desired enantiomer by salt formation can be avoided. The surprising findings of the present invention makes it feasible that the overall ramelteon synthesis from readily available starting compound involves only six steps, and from a chosen intermediate compound 6,7-dihydro-1H-indeno[5,4-b]furan-8(2H)-one (THI; corresponding to the compound of formula V) involves even only 3 steps in comparison to procedure of 5 steps, described in Tetrahedron Asymmetry 17, 2084 (2006), while the associated benefits of short and efficient synthesis route are unmet in prior art processes. Moreover, the present invention provides a novel intermediate compound useful for contributing a relevant and enantioselective structural moiety to the final compound defined by the structure of ramelteon or analogues thereof.

### DETAILED DESCRIPTION OF THE INVENTION

In the following, the present invention will be described in more detail by preferred embodiments and examples noting, however, that these embodiments, examples are presented for illustrative purposes only and shall not limit the invention in any way.

Reaction Scheme 1 illustrates a preferred embodiment of the process according to present invention for preparing ramelteon (VIIIa).

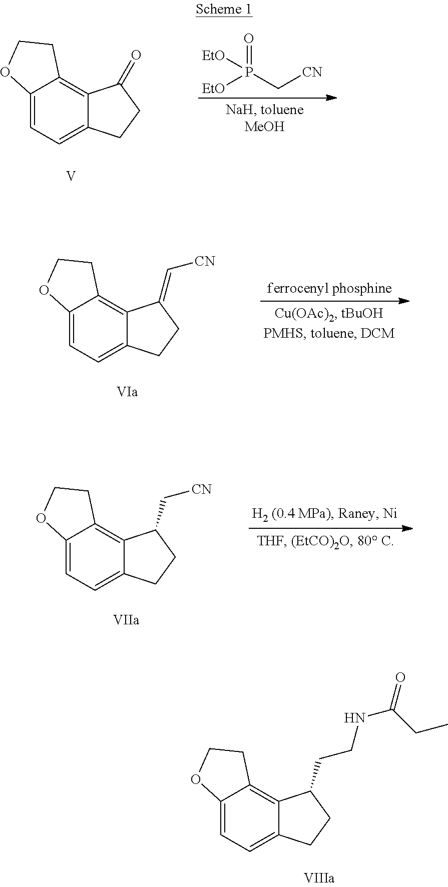

According to the preferred embodiment of Scheme 1 compound of formula VIa is prepared by reacting compound of formula V with cyanomethanephosphonate of formula (R³O)₂POCH₂CN. While R³ in reaction Scheme 1 is ethyl, other groups are possible, for example selected from the group consisting of substituted or unsubstituted alkyl, cycloalkyl and arylalkyl group. Preferably diethyl cyanomethanephosphonate is used, preferably in an equimolar ratio to compound of formula V or in a slight excess. The reaction to compound VIa is carried out in the presence of a base such as NaH or sodium methoxide. Reaction is performed in organic solvent, preferably in toluene or methanol.

Further according to the preferred embodiment illustrated by Scheme 1, compound of formula VIIa is prepared by subjecting a compound of formula VIa to asymmetric reduction in the presence of catalysts selected from complexes comprising transition metals, preferably Cu, Co, Ni, Rh, Ru, Pd, Ir and chiral ligands, Preferably catalyst is metal-(optically active posphine)-complex, wherein metal is preferably Cu, Rh, Ru, Pd, Ir, more preferably Cu, wherein said catalyst particularly is a copper-(optically active phosphine)-complex catalyst.

Asymmetric reduction can be advantageously carried out under normal or atmospheric pressure, applying a suitable hydride source. Preferable hydride source is a gentle hydride donor source, in particular polymethylhydrosiloxane (PMHS). According to particularly beneficial and efficient embodiment, said metal-(optically active phosphine)-complex catalyst is prepared in situ from the corresponding metal, preferably copper, (as source of metal preferably the corresponding metal acetate or [(PPh₃)₃(metal)H]₆, is used, more preferably metal acetate is used, wherein aforementioned “metal” preferably is copper), and ferrocenyl phosphines selected from the group of compounds having formula:

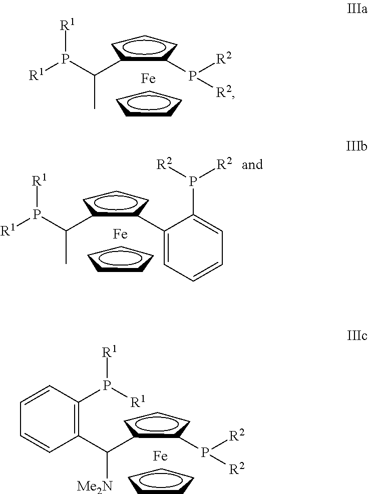

wherein R¹ and R² are independently selected from the group consisting of independently substituted or unsubstituted alkyl, cycloalkyl, aryl heteroaryl, arylalkyl and heteroarylalkyl group. Preferably compound of formula IIIa, wherein R¹ is cyclohexyl and R² is phenyl is used. The reaction is preferably performed under inert atmosphere. To ferrocenyl phosphine and corresponding metal source such as copper acetate, an organic solvent, preferably toluene, is added. The solution is subsequently cooled below room temperature, preferably at around 0° C. A hydride source, preferably polymethylhydrosiloxane (PMHS), is added, preferably in molar excess compared to compound VIa. Then compound of formula VIa is added followed by addition of t-BuOH, preferably in molar excess compared to compound VIa. After stirring the reaction mixture for a suitable period of time, for example for at least 30 minutes and preferably for about 1 hour, dichloromethane is added and reaction mixture is heated, preferably to a temperature from 10° C. to 80° C., more preferably to about room temperature such as about 20° C. to 25° C. Reaction mixture is left to stir for a further suitable period of time, for example 2 to 22 hours, preferably for about 15 hours at the last mentioned temperature, as noted preferably at about room temperature. Reaction may then be stopped, for example by subsequently adding NaOH (preferably 1N NaOH/10% NaCl solution). If desired to provide compound VIIa in isolated form, extractive work up furnishes crude compound VIIa with high yields (>80%) and high chemical as well as enantiomeric purity (87% to 96% ee). Optionally further purification can be performed, preferably by chiral HPLC, to yield compound VIIa of still higher enantiomeric purity.

Alternatively, compound VIIa may be provided, after completion of the previously described reaction, in non-isolated form without further extractive work and may as such be subjected to further synthesis reactions as described herein.

Further according to the preferred embodiment of Scheme 1, compound of formula VIIIa is prepared from a compound of formula VIIa by converting the cyano group of the compound of formula VIIa into propanamide group bonded to the carbon atom of the cyano group to give the compound of formula VIIIa. This conversion of cyano group of the compound of formula VIIa into propanamide group bonded to the carbon atom of the cyano group to give the compound of formula VIIIa is preferably carried out with hydrogen and propionic anhydride in the presence of suitable catalyst in one step. In prior art processes two steps are needed for said conversion and isolation of a compound of formula IX due to purification purposes is required. According to present invention said isolation step can be omitted.

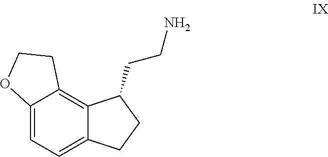

A suitable catalyst may be composed of nickel which optionally may be mixed with aluminium or cobalt or both, a preferred catalyst is Raney-Ni. The conversion of the cyano group into propanamide group bonded to the carbon atom of the cyano group can be realized under relatively low hydrogen pressure conditions, such as ≦10 MPa and more preferably ≦0.5 MPa.

Specific and preferred conditions for the conversion reaction can be set as follows: First Raney-Ni in water is added to reaction vessel. Subsequently organic solvent is added. Preferably aprotic organic solvent is used, more preferably aprotic organic solvent is tetrahydrofurane (THF). Compound of formula VIIa and propionic anhydride are added to organic solvent. Propionic anhydride is preferably used in molar excess of more then 2 molar equivalents, more preferably more then 5 molar equivalents, most preferably more than 10 molar equivalents, compared to compound VIIa. The reaction is performed in the presence of hydrogen at temperature from 20° C. to 120° C., preferably at about 80° C. The reaction mixture is left to stir for 1 to 24 hours, preferably for about 10 hours.

Reaction mixture is then cooled down, preferably to about room temperature such as about 20° C. to 25° C. and filtered. Solution is diluted, preferably with toluene, and water phase, preferably solution of NaOH, is added. Further extractive work up furnish crude ramelteon (VIIIa) which is isolated or recovered from the organic phase by precipitation or crystallization.

The precipitation or crystallization is preferably caused by adding an antisolvent, e.g. water, ethers and hydrocarbons. Preferably hexane is used as antisolvent.

Optionally, further purification can be performed by recrystallization, reprecipitation, slurrying, optionally by HPLC, to yield compound VIIIa of still higher purity.

The compound 6,7-dihydro-1H-indeno[5,4-b]furan-8(2H)-one (V), from which synthesis Scheme 1 above started, is available; for example it can be prepared by synthesis routes known to a person skilled in the art.

According to the preferred manner the compound of formula V is prepared according to a process illustrated in reaction Scheme 2

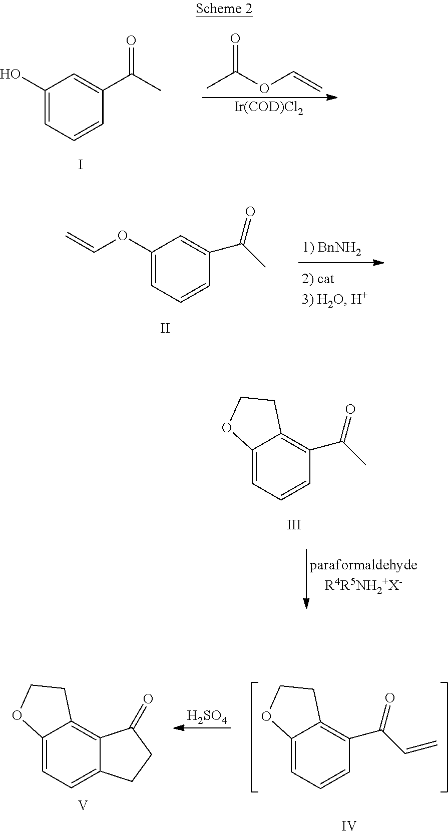

According to the preferred way of making compound V available as illustrated by Scheme 2, compound of formula II is prepared by protecting a compound of formula I with vinyl group. Preferably vinyl acetate in the presence of Ir(COD)Cl)2 is used. The reaction is preferably performed at about 50° C. to 120° C. for 2 to 4 hours.

Further according to the preferred way of making compound V available as illustrated by Scheme 2, compound of formula III is prepared by reacting a compound of formula II with primary amine, preferably benzylamine. The reaction is preferably performed in the presence of a catalyst, preferably selected from the group consisting of metal catalyst, such as for example rhodium or ruthenium, or from derivative of said metal, such as for example Cp* or phosphines.

The reaction is preferably performed at about 50° C. to 200° C. for, more preferably at about 100° C. to 180° C., most preferably at about 140° C. to 160° C.

Further according to the preferred way of making compound V available as illustrated by Scheme 2, a compound of formula III is reacted with paraformaldehyde in the presence of an ammonium salt of formula R⁴R⁶NH₂⁺X⁻, (wherein R⁴ and R⁵ are each independently selected from alkyl, cycloalkyl, aryl, arylalkyl and arylcycloalkyl; and X is halogen, BF₄, PF₆, H₂PO₄ or R⁶CO₂, wherein R⁶ is one of alkyl, aryl, polyhaloalkyl) [wherein “alkyl” may preferably denote C₁ to C₆ alkyl], such as for example TADCA or TAMA.

The excess of the ammonium salt (up to 1 equivalent) can be used.

The reaction is preferably performed in aprotic solvent for 1 to 36 hours, more preferably for 4 to 12 hours, at about 60° C. to 120° C.

At this stage acrylate intermediate IV can be effectively obtained in the form of a solution in organic solvent. The organic solvent is suitably an apolar solvent and is preferably selected from the group of alkanes, ethers or chlorinated solvents. Advantageously, it is not necessary that intermediate IV is isolated.

The solution is then reacted with strong inorganic acid, preferably sulfuric acid, at a temperature between 0° C. to 100° C., preferably 30° C. to 70° C. to give a compound of formula V.

Another aspect of the invention is a process for preparing ramelteon (VIIIa) comprising the step of:

a.) providing a compound of formula VI:

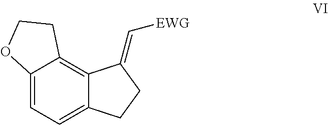

wherein EWG is an electron withdrawing group; and

b.) performing asymmetric reduction of the compound of formula VI in the presence of metal (optically active posphine) complex catalyst, wherein metal is preferably Cu, Co, Ni, Rh, Ru, Pd or Ir, more preferably Cu, wherein said catalyst particularly is a metal-(optically active phosphine) complex catalyst of the aforementioned metal, preferably copper, to give the compound of formula VII

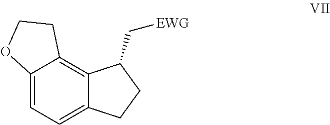

This aspect of the present invention renders the concept of the present invention to be applicable more generally, while still enabling an enhanced and efficient synthesis route as desired to eventually yield ramelteon (VIIIa). In particular, while it is most preferred that the electron withdrawing group is cyano in terms of providing significantly advanced further synthesis steps, the electron withdrawing group may also be selected from the group consisting of halogens (F, Cl, Br and I, preferably F and Cl), carboxylic acid (CO₂H), carboxylic acid esters (CO₂R⁷, wherein R⁷ is selected from the group consisting of substituted or unsubstituted alkyl, cycloalkyl and arylalkyl group) and amides (CONR⁸R⁹, wherein R⁸ and R⁹ are the same or different and respectively denote H, substituted or unsubstituted alkyl, cycloalkyl [wherein “alkyl” preferably denotes C₁ to C₆ alkyl], preferably R⁸ and R⁹ are both H).

For specific alternative embodiments of this aspect of the present invention, the phosphonate compound illustrated in the above Scheme 1 may be bonded to a cyano group or, correspondingly, to the respective other organic groups representing an alternative electron withdrawing group (EWG) to be used instead of cyano. Using phosphonoacetic esters, compounds of formula VI and VII wherein EWG is CO₈R⁷ can be obtained, wherein said CO₂R⁷ group can subsequently be converted to CO₂H group by hydrolysis. Using phosphonoacetamides, compounds VI and VII wherein EWG is CONR⁸R⁹ can be obtained, in which R⁷, R⁸, R⁹ are the same as above. Using halomethanophosphonates, compounds of formula VI and VII wherein EWG is F, Cl, Br or I can be obtained.

For performing the asymmetric reduction, reference can be made to the above description about the conversion from compound VIa to compound VIIa. For example, said asymmetric reduction can be advantageously performed in the presence of hydride source, preferably polymethylhydrosiloxane (PMHS) and wherein said metal-(optically active phosphine)-complex catalyst is prepared from the aforementioned metal, preferably copper (as source of metal preferably metal acetate or [(PPh₃)₃(metal)H]₆, is used, more preferably metal acetate is used, wherein “metal” is as defined above, most preferably copper) and ferrocenyl phosphines selected from the compounds having formula:

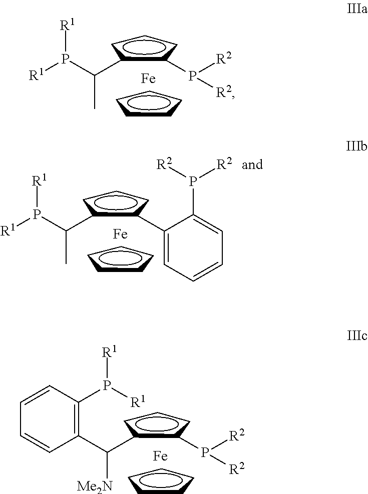

wherein R¹ and R² are independently selected from the group consisting of independently substituted or unsubstituted alkyl, cycloalkyl, aryl heteroaryl, arylalkyl and heteroarylalkyl group. Preferably compound of formula IIIa, wherein R¹ is cyclohexyl and R² is phenyl is used.

After the asymmetric reduction step the resulting compound of formula VII can then be subjected to further synthetic steps to yield ramelteon (VIIIa). If EWG in formula VII is halogen a further transformation can be performed by the substitution of halogen with cyanide, preferably reacting with alkali metal cyanide and further transformed the obtained cyano derivative VIIa as described above. If EWG is CONH₂ a further transformation can be performed by reduction to the compound IX and subsequent propanoylation to yield ramelteon (VIIIa). If EWG is CO₂R⁷ a further transformation can be performed by conversion of ester to amide (CONH₂,) and conversion of said amide to ramelteon (VIIIa) as described above. If EWG is CONR⁸R⁹ and R⁸, R⁹ are removable groups the CONR⁸R⁹ group is first transformed to CONH₂ group and from there ramelteon (VIIIa) is prepared as described above. If EWG is CONR⁸R⁹ and R⁸, R⁹ cannot be removed the further synthesis steps could be applied to obtain derivatives similar to ramelteon (VIIIa).

In another aspect the present invention relates to a process for preparing the compound of formula VIII

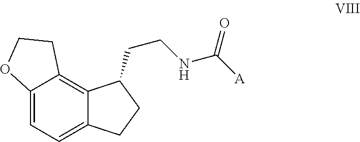

wherein A is selected from the group consisting of linear C₁-C₅-alkyl, branched C₁-C₅-alkyl, ethenyl and ethynyl, comprising the steps of:
a.) providing a compound of formula VIIa

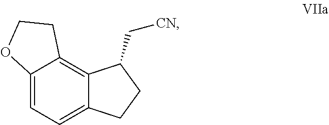

and
b.) converting the cyano group of the compound of formula VIIa into C₁-C₅-alkanamide, propenamide, propynamide group, bonded to the carbon atom of the cyano group to give the compound of formula VIII.

Said process can be used to prepare analogues of ramelteon (VIIIa) and is performed in a analogous way as described above for ramelteon (VIIIa). Instead of propionic anhydride other linear or branched C₁-C₅-alkananhydrides, acryl anhydride, propargyl anhydride, mixtures of acetanhydride and C₁-C₅-alkanoic acids, acrylic or propargylic acid are used to prepare compounds of formula VIII analogous to ramelteon (VIIIa).

According to preferred embodiments, the process can be made particularly efficient for the synthesis of ramelteon.

For preparing a pharmaceutical composition comprising ramelteon (VIIIa) as active ingredient, first ramelteon (VIIIa) is provided by the processes as disclosed herein, and then the thus prepared ramelteon (VIIIa) is admixed with at least one suitable pharmaceutically acceptable excipient. Pharmaceutically acceptable excipients may be selected from the group consisting of binders, diluents, disintegrating agents, stabilizing agents, preservatives, lubricants, fragrances, flavoring agents, sweeteners and other excipients known in the field of the pharmaceutical technology. Preferably, carriers and excipients may be selected from the group consisting of lactose, microcrystalline cellulose, cellulose derivatives, e.g. hydroxyl)propylcellulose, polyacrylates, calcium carbonate, starch, colloidal silicone dioxide, sodium starch glycolate, talc, magnesium stearate, polyvinylpyrrolidone, polyethylene glycol and other excipients known in the field of the pharmaceutical technology.

### Experimental Procedures

### EXAMPLE 1

### Preparation of 1-(3-(vinyloxy)phenyl)ethanone (II)

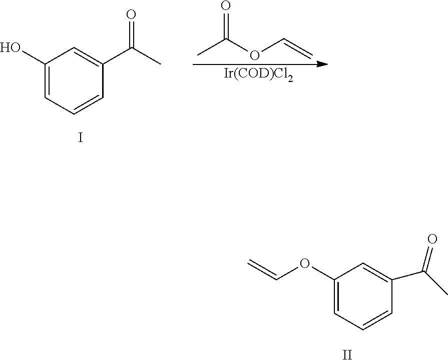

1-(3-hydroxyphenyl)ethanone (I) (5 g, 36.8 mmol) was suspended in dry toluene (37 ml), dry sodium carbonate (2.34 g, 0.6 eq) and (Ir(COD)Cl)₂ (247 mg, 0.1 eq) were added. Vinyl acetate (6.8 ml, 2 eq) was finally added and the reaction was heated at 100° C. for 2 h. Reaction was cooled down to room temperature, filtered and concentrated. Residue was purified by flash chromatography (100% hexane to 95/5 hexane/EtOAc) to give 1-(3-(vinyloxy)phenyl)ethanone (II) (5.05 g, 85%). ¹H NMR δ (CDCl₃) 7.65 (d, 1H, J=7.7 Hz), 7.56 (t, 1H, J=2.0 Hz), 7.40 (t, 1H, J=8.0 Hz), 7.19 (dd, 1H, J=2.5 Hz, J=8.1 Hz), 6.66 (dd, 1H, J=6.0 Hz, J=13.7 Hz), 4.80 (dd, 1H, J=1.8 Hz, J=13.7 ), 4.50 (dd, 1H, J=1.8 Hz, J=6.0 Hz), 2.58 (s, 3H). ¹³C NMR δ (CDCl₃) 197.3, 156.9, 147.5, 138.6, 129.8, 123.1, 121.8, 116.0, 96.1, 26.6.

### EXAMPLE 2

### Preparation of 1-(2,3-dihydrobenzofuran-4-yl)ethanone

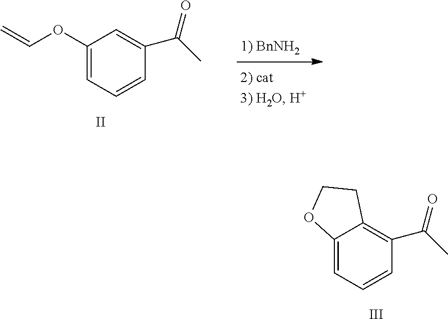

1-(3-(vinyloxy)phenyl)ethanone (II) (1.62 g, 10 mmol) was dissolved in dry toluene (100 ml), 4 Å molecular sieves (10 g, 1 g/mmol) and benzylamine (1.1 ml, 10 mmol) were added and the reaction was heated at reflux for 18 h. Reaction was cooled down to room temperature, filtered and concentrated. Residue was dissolved in toluene (100 ml), Ph₃PRhCI (462 mg, 0.05 eq) was added and reaction was heated for 24 h at 150° C. in a pressure reactor. Reaction was cooled down to room temperature, 1N HCl (100 ml) was added and the reaction was stirred for 2 h. Phases were separated and organic phase was washed successively with 1N HCl, water and brine. Organic phase was dried over MgSO₄, filtered, concentrated and purified by flash chromatography to give 1-(2,3-dihydrobenzofuran-4-yl)ethanone (III) (1.17 g, 72%). ¹H NMR δ (CDCl₃) 7.35 (dd, 1H, J=0.8 Hz, J=7.8 Hz), 7.19 (t, 1H, J=7.9 Hz), 6.95 (d, 1H, J=8.0 Hz), 4.57 (t, 2H, J=8.8 Hz), 3.52 (t, 2H, J=8.8 Hz), 2.57 (s, 3H). ¹³C NMR δ (CDCl₃) 198.8, 161.0, 133.8, 128.2, 127.9, 121.4, 113.4, 71.6, 31.0, 27.6.

### EXAMPLE 3

### Preparation of 6,7-dihydro-1H-indeno[5,4-b]furan-8(2H)-one (V)

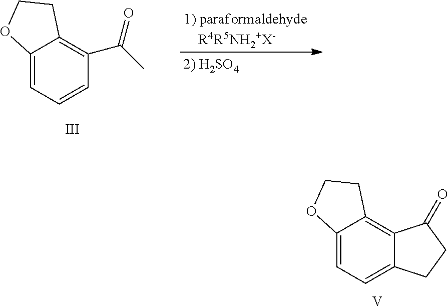

1-(2,3-dihydrobenzofuran-4-yl)ethanone (III) (1 g, 6.2 mmol) was dissolved in dioxane (9 ml). TADCA (dicyclohexylammonium 2,2,2-trifluoroacetate) (1.82 g, 1 eq) and paraformaldehyde (0.611 g, 1.1 eq) were added. The reaction was heated at 100° C. for 2 h. A second portion of TADCA (0.91 g, 0.5 eq) and paraformaldehyde (0.333 g, 0.6 eq) were added and the reaction was heated at 100° C. for 2 h. Reaction was partitioned between water (20 ml) and pentane (30 ml). Aqueous phase was re-extracted 4 times with pentane (10 ml). Combined pentane phases were washed with water and brine, dried over MgSO₄. Solution was diluted to 100 ml with pentane. This solution was added dropwise to a pre-heated solution of sulfuric acid at 67° C. (10 ml) under nitrogen stream. At the end of addition, the reaction was stirred for 30 min. Reaction was cooled down to room temperature and poured on iced water (50 ml). Solution was extracted 5 times with MTBE. Combined organic phases were washed with water, NaHCO₃ 1M and brine, dried over MgSO₄ and concentrated. Purification by flash chromatography furnished pure 6,7-dihydro-1H-indeno[5,4-b]furan-8(2H)-one (V). ¹H NMR δ (CDCl₃) 7.21 (dd, 1H, J=0.9 Hz, J=9.0 Hz), 7.02 (d, 1H, J=8.2 Hz), 4.66 (t, 2H, J=8.9 Hz), 3.48 (t, 2H, J=8.9 Hz), 3.08 (dd, 2H, J=4.9 Hz, J=6.0 Hz), 2.69 (m, 2H). ¹³C NMR S (CDCl₃) 207.5, 160.2, 147.1 133.6, 125.6, 123.9, 115.6, 72.3, 37.1, 28.4, 25.4.

### EXAMPLE 4

### Preparation of (E)-(1,6,7,8-Tetrahydro-2H-indeno[5,4-b]furan-8-ylidene)acetonitrile (VIa)

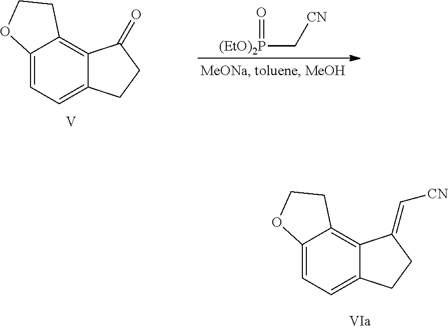

NaH (0.5 g, 1.2 eq) was suspended into a solution of toluene (15 ml), reaction was cooled down to 0° C. Diethyl cyanomethanephosphonate (2 ml, 1.2 eq) was added and the reaction was stirred for 1 hour. 6,7-dihydro-1H-indeno[5,4-b]furan-8(2H)-one (1.8 g, 10.3 mmol) in suspension in toluene (25 ml) was added slowly. MeOH (0.5 ml) was added and the reaction was warmed up to room temperature and stirred for 3 hours. Reaction was quenched with water (25 ml). Phases were separated, aqueous phase was re-extracted with toluene (15 ml). Combined organic phases were washed with brine, dried over MgSO₄ and concentrated. Purification by flash chromatography gave compound VIa (amount 1.56 g, yield 76.5%).

### EXAMPLE 5

### Preparation of (S)-2-(2,6,7,8-tetrahydro-1H-indeno[5,4-b]furan-8-yl)acetonitrile (VIIa)

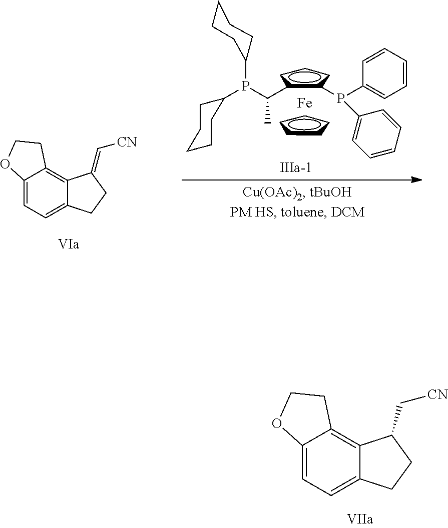

In a dry flask under inert atmosphere, were added catalyst IIIa-1 (32 mg) and copper acetate (9 mg, 0.1 eq), followed by toluene (5 ml). Solution was cooled at 0° C. polymethylhydrosiloxane (PMHS) (1.26 ml, 4 eq) was added and the reaction was stirred for 5 to 10 min. Compound VIa (1 g, 5 mmol) in solution in toluene (5 ml) was added, followed by tBuOH (1.9 ml, 4 eq). Reaction was stirred for 45 min at 0° C., dichloromethane (DCM) was added (0.5 ml) and the reaction was slowly warmed up to room temperature. Reaction was stirred for 15 h. NaOH 1N/10% NaCl solution (10 ml) was added and the reaction was stirred for 30 min. Phases were separated and aqueous solution was re-extracted twice with MTBE. Combined organic phases were dried over MgSO₄ and concentrated. Purification by flash chromatography furnished pure compound VIIa in 87% ee.

¹H NMR δ (CDCl₃) 6.99 (d, 1H, J=8 Hz), 6.67 (d, 1H, J=8 Hz), 4.66-4.52 (m, 2H), 3.51 (m, 1H), 3.30 (m, 1H), 3.17 (m, 1H), 2.98 (m, 1H), 2.83 (m, 1H), 2.69 (dd, 1H, J=16.8 Hz, J=5.3 Hz), 2.54 (dd, 1H, J=16.8 Hz, J=8.3 Hz), 2.44 (m, 1H), 2.00 (m, 1H). Mass (m+1) 175

### EXAMPLE 6

### Preparation of (S)—N-(2-(2,6,7,8-tetrahydro-1H-indeno[5,4-b]furan-8-yl)ethyl)propionamide-ramelteon (VIIIa)

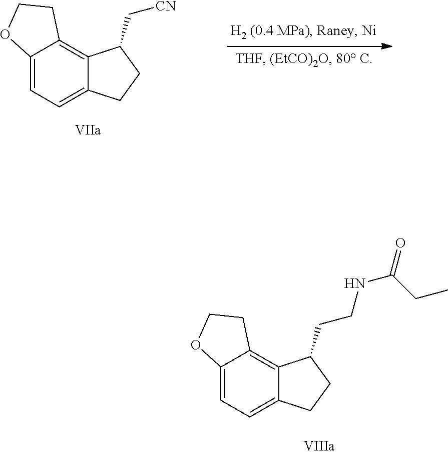

Raney-Ni in water was added to the reaction vessel and was washed 5 times with absolute EtOH and 4 times with dry THF. THF (11 ml) was then added. Compound VIIa (200 mg, 1 mmol) was added, followed by propionic anhydride (1.5 ml, 11.5 eq). Reactor was sealed and filled with hydrogen (0.4 MPa). Reaction was heated at 80° C. and stirred overnight. Reaction was then cooled down to room temperature and filtered on celite©. Solution was diluted with toluene (20 ml) and NaOH 2N (10 ml) and reaction was stirred for 30 min. Phases were separated, organic phase was washed with NaOH 2N (10 ml) and brine. Solution was dried over MgSO₄ and concentrated. Solid was dissolved in EtOAc (2 ml) and hexane (20 ml) was slowly added to promote crystallization. Solid was filtered to give pure Ramelteon (VIIIa) (210 mg, 81%). ¹H NMR δ (CDCl₃) 6.94 (d, 1H, J=7.9 Hz), 6.60 (d, 1H, J=7.9 _{Hz),} 5.65 (br s, 1H), 4.60-4.46 (m, 2H), 3.31 (q, 2H, J=6.8 Hz), 3.28-3.05 (m, 3H), 2.88 (m, 1H), 2.76 (m, 1H), 2.27 (m, 1H), 2.18 (q, 2H, J=7.6 Hz), 2.01 (m, 1H), 1.82 (m, 1H), 1.63 (m, 1H), 1.14 (t, 3H, J=7.6 Hz). ¹³C NMR δ (CDCl₃) 173.7, 159.2, 143.0, 135.7, 123.3, 122.1, 107.3, 71.1, 42.1, 37.9, 33.3, 31.6, 30.6, 29.7, 28.5, 9.8.

1. A process for preparing the compound of formula VIII 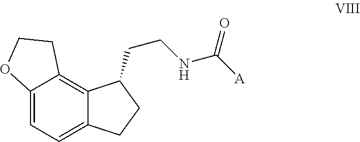
wherein A is selected from the group consisting of linear C₁-C₅-alkyl, branched C₁-C₅-alkyl, ethenyl and ethynyl, the process comprising the steps of:
a.) providing a compound of formula VIIa 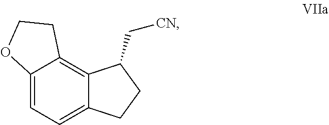
and
b.) converting the cyano group of the compound of formula VIIa into C₁-C₅-alkanamide, propenamide or propynamide group bonded to the carbon atom of the cyano group, by reaction of the compound of formula VIIa with a corresponding reactant selected from the group consisting of C₁-C₅-alkananhydrides; acryl anhydride; propargyl anhydride; and mixtures of acetanhydride and C₁-C₅-alkanoic acids, acrylic or propargylic acid, to give the respective compound of formula VIII.

2. The process according to claim 1, wherein A is selected from the group consisting of linear C₁-C₅-alkyl and branched C₁-C₅-alkyl, and wherein in step b.) the reaction of the compound of formula VIIa is correspondingly carried out with a linear or branched C₁-C₅-alkananhydride.

3. The process according to claim 1, wherein conversion step b.) comprises reduction.

4. The process according to claim 3, wherein the reduction comprises using hydrogen in the presence of catalyst, preferably wherein the catalyst is Raney-Ni.

5. The process according to claim 3, wherein in step b.) reduction is carried out under hydrogen pressure conditions of ≦0.5 MPa.

6. A process according to claim 1, for preparing the ramelteon with formula VIIIa 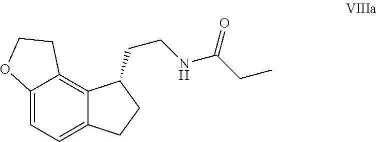
the process comprising the steps of:
a.) providing a compound of formula VIIa 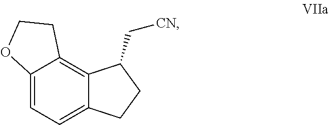
and
b.) converting the cyano group of the compound of formula VIIa into propanamide group bonded to the carbon atom of the cyano group to give the compound of formula VIIIa.

7. The process according to claim 1, wherein step b.) comprises both a reaction of the compound of formula VIIa with said corresponding reactant and a reduction to be performed in one pot to give the respective compound of formula VIII or VIIIa.

8. A process for preparing a compound of formula VII comprising the steps of:
a.) providing a compound of formula VI: 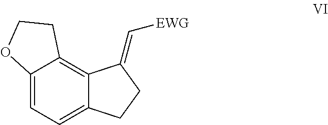
wherein EVVG means an electron withdrawing group; and
b.) performing asymmetric reduction reaction of the compound of formula VI in the presence of metal-(optically active posphine)-complex catalyst, wherein metal is preferably selected from the group consisting of Cu, Co, Ni, Rh, Ru, Pd and Ir, to give the compound of formula VII:

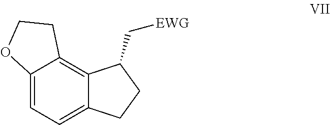

9. The process according to claim 8, wherein asymmetric reduction is performed in the presence of hydride source, preferably using polymethylhydrosiloxane.

10. The process according to claim 8, wherein said metal-(optically active phosphine)-complex catalyst is prepared from corresponding metal, copper and ferrocenyl phosphines selected from the compounds having formula: 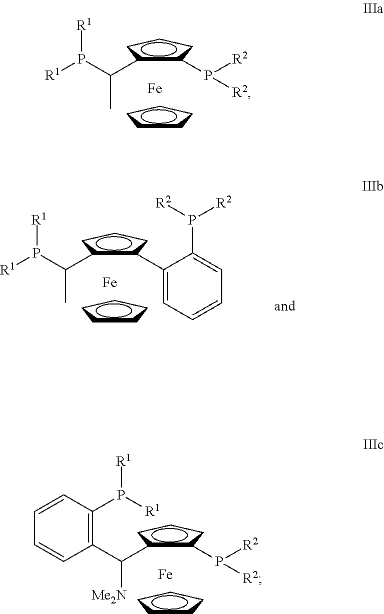
wherein R¹ and R² are independently selected from the group consisting of independently substituted or unsubstituted alkyl, cycloalkyl, aryl heteroaryl, arylalkyl and heteroarylalkyl group.

11. The process according to claim 8, further comprising subjecting the compound of formula VII to further synthetic steps to yield a compound having the formula VIII 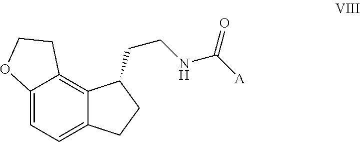
wherein A is selected from the group consisting of linear C₁-C₅-alkyl, branched C₁-C₅-alkyl, ethenyl and ethynyl,

12. The process according to claim 11, wherein the further synthetic steps yield ramelteon (VIIIa).

13. The process according to claim 8, wherein the EWG group is selected from nitrile (CN), halogens F, Cl, Br and I, carboxylic acid (CO₂H), carboxylic acid esters (CO₂R⁷, wherein R⁷ is selected from the group consisting of substituted or unsubstituted alkyl, cycloalkyl and arylalkyl group) and amides (CONR⁸R⁹, wherein R⁸ and R⁹ are the same or different and respectively denote H, substituted or unsubstituted alkyl, cycloalkyl, wherein “alkyl” may denote C₁ to C₆ alkyl.

14. The process according to claim 13, wherein the compound of formula VI is prepared by a process comprising reacting compound of formula V 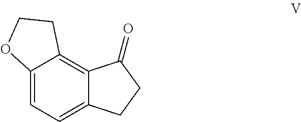
with a compound of formula (R³O)₂POCH₂(EWG), wherein EVVG is CN and R³ is selected from the group consisting of substituted or unsubstituted alkyl, cycloalkyl and arylalkyl group.

15. The process according to claim 14 wherein the compound of formula V is prepared by a process comprising the steps of:
a.) preparing a compound of formula II by reacting compound of formula I with vinyl acetate;
b.) preparing a compound of formula III by reacting a compound of formula II with primary amine;
c.) reacting a compound of formula III with paraformaldehyde in the presence of ammonium salt, R⁴R⁵NH₂⁺X⁻, (wherein R⁴ and R⁵ are each independently selected from alkyl, cycloalkyl, aryl, arylalkyl and arylcycloalkyl; and X is halogen, BF₄, PF₆, H₂PO₄ or R⁶CO₂, wherein R⁶ is one of alkyl, aryl, polyhaloalkyl) in organic solvent,
d.) contacting the solution from step c) with strong inorganic acid

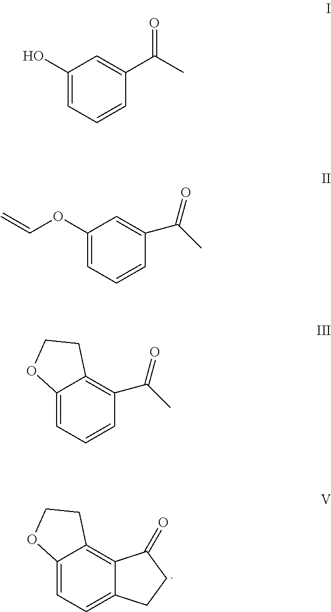

16. A compound of formula VIIa

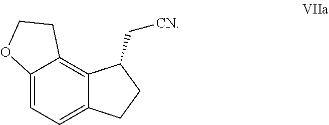

17. (canceled)

18. (canceled)

19. A process for preparing ramelteon (VIIIa) comprising the steps of:
a.) preparing the compound of formula VIIa by a process comprising subjecting a compound of formula VIa to asymmetric reduction in the presence of the copper-(optically active phosphine)-complex catalyst and hydride source, wherein said copper-(optically active phosphine)-complex catalyst is prepared from copper and ferrocenyl phosphines selected from the compounds having formula: 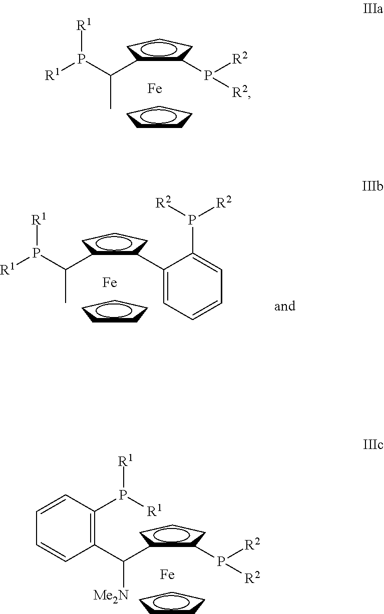
wherein R¹ and R² are independently selected from the group consisting of independently substituted or unsubstituted alkyl, cycloalkyl, aryl heteroaryl, arylalkyl and heteroarylalkyl group; and
b.) reacting a compound of formula VIIa with hydrogen and propionic anhydride in the presence of catalyst, wherein said catalyst is Raney-Ni, to yield a compound of formula VIIIa

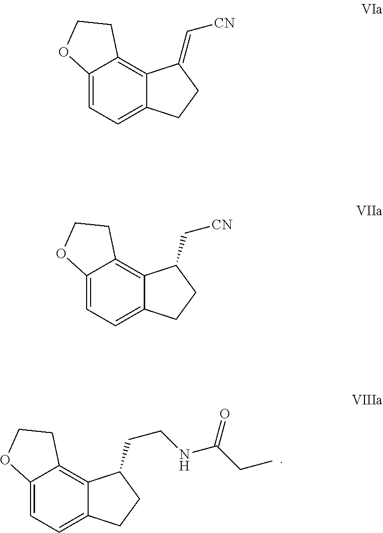

20. The process according to claim 19, wherein the compound of formula VIa has previously been obtained by reacting compound of formula V with cyanomethanephosphonate of formula (R³O)₂POCH₂CN, wherein R³ is selected from the group consisting of substituted or unsubstituted alkyl, cycloalkyl and arylalkyl group; to yield a compound of formula VIa

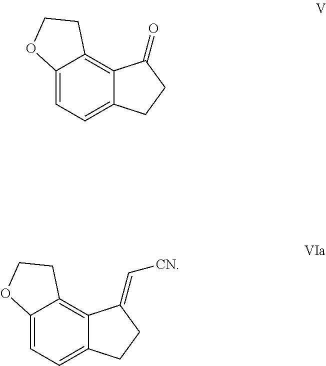

21. A process according to claim 20, wherein the compound of formula V has previously been obtained by
a.) preparing a compound of formula II by reacting compound of formula I with vinyl acetate;
b.) preparing a compound of formula III by reacting a compound of formula II with primary amine;
c.) reacting a compound of formula III with paraformaldehyde in the presence of ammonium salt, R⁴R⁵NH₂⁺X⁻ (wherein R⁴ and R⁵ are each independently selected from alkyl, cycloalkyl, aryl, arylalkyl and arylcycloalkyl; and X is halogen, BF₄, PF₆, H₂PO₄ or R⁶CO₂, wherein R⁶ is one of alkyl, aryl, polyhaloalkyl) in organic solvent;
d.) contacting the solution from step c.) with strong inorganic acid and obtaining compound of formula V;

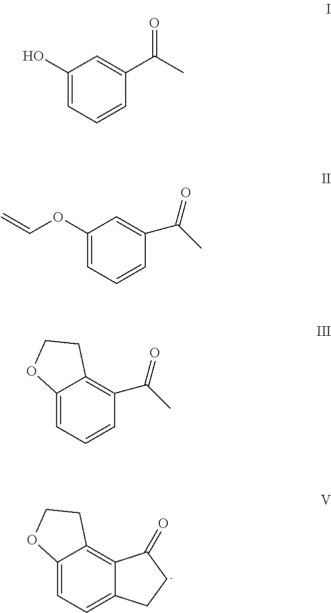

22. A process for the preparation of a pharmaceutical composition comprising ramelteon (VIIIa) as active ingredient, comprising the steps of:
preparing ramelteon (VIIIa) according to the process according to any one of the claims 6, 7 and 12-15 and 19-21, and
admixing the thus prepared ramelteon (VIIIa) with at least one pharmaceutically acceptable excipient.
